(19) United States
(12) Patent Application Publication
(10) Pub. No.: US 2005/0101990 A1
(43) Pub. Date: May 12, 2005

(54) Wire dispenser for use with forceps

(76) Inventors: Steven B. Aragon, Englewood, CO (US); James W. Heller, Boulder, CO (US)

Correspondence Address: Ruth Eure, 4795 Edison Avenue, Boulder, CO, 80301, US

(21) Appl. No.: 10/223,095

(22) Filed: Aug. 19, 2002

(51) Int. Cl. A61B 17/28

(52) U.S. Cl. 606/205; 206/63.3

(57) ABSTRACT

The invention provides a wire dispensing device for use with forceps which attaches to a pair of forceps and provides a continuous source of wire or suture material for surgical procedures and means for covering the exposed end of the wire.

### FIELD OF THE INVENTION

The present invention relates to the field of surgical instruments, and more particularly to the field of orthopedic, maxillofacial and reconstructive surgical instruments.

### BACKGROUND OF THE INVENTION

Broken or shattered bones are sometimes treated by stabilizing the bone fragments by the method of wiring the bone fragments to a support structure, or to other bones. The wiring method commonly used requires passing pieces of wire, typically 4-10 inches cut from spools from a surgical assistant to the surgeon. This procedure is problematic because the wire ends are exposed and can perforate surgical gloves. The 22-26 gauge wire easily punctures surgical gloves and skin. Puncture wounds occur when the wire is passed, twisted and cut. This problem is particularly dangerous if the patient has an infectious disease such as AIDS. This problem has been described in the medical literature. See, for example, Avery, C. M. Johnson, P. A., “Surgical glove perforation and maxillofacial trauma: To plate or wire?” Br. J. Oral Maxillofac. Surg., vol. 30, no. 1, pp. 31-35 1992, and Avery, C. M. E., Taylor, J. and Johnson, P. A., “Double gloving and a system for identifying glove perforations in maxillofacial trauma surgery,” Br. J. Oral Maxillofac. Surg., vol. 37, no. 4, pp. 316-319 1999. The present invention provides a device and method which largely eliminates the perforation problem due to exposed wire ends.

A broken jaw or mandibular fracture is commonly treated by stabilizing the bone fragments. Typically, a metal bar called an arch bar is attached to the teeth using wire. The arch bar is placed on the outside surface of the teeth. Solid stainless steel wire is wrapped around each tooth and twisted around the arch bar. The twisted end of the wire is bent back over onto itself. Alternatively, the bone fragments can be wired directly to each other. This arrangement stabilizes the Jaw during the healing process. This treatment is also used to stabilize bone ‘fragments in orthopedic and reconstructive surgery.

Typically, the wire is handled and twisted using forceps. The prior art reflects several techniques for accomplishing this task.

For example, the Corwin Automatic Twisting Forceps utilize engagement of a wire with a rod having a spiral thread. By pulling the ring on the end of the rod, the wires are twisted as the rod rotates.

U.S. Pat. No. 3,759,302 to Attenborough describes a wire twisting device that utilizes wire guides and a screw drive mechanism.

U.S. Pat. No. 4,656,860 to Orthuber et al describes an electrically driven device to bend and twist wire pieces.

U.S. Pat. No. 4,903,826 discusses a dispenser for surgical guidewire. This invention is designed for inserting catheters into arteries, and bears minimal relevance to the objects of the present invention. Also, this patent contains no mention whatsoever of fastening the device to forceps.

U.S. Pat. No. 1,380,433 for a dental wire dispenser and mounting tool is designed for dental pins used for dental implants. The description of this device requires use of small segments of wire and therefore bears no similarities to the instant invention, and in fact, misses the point of the present invention.

U.S. Pat. No. 6,093,179, is directed to a guide wire dispenser for a feeding tube. This bears no relevance to the instant invention since a significant point of novelty in the instant invention is that it is specifically adapted for use with forceps. The '179 patent contains no suggestion of substituting surgical wire, nor is there any indication that the '179 device can be used with forceps to manipulate the wire, such as is the case with the instant invention.

U.S. Pat. No. 3,995,628 is directed to a catheter insertion device. This is a hand-held spool for inserting a catheter into a patient. This disclosure contains no mention of wire, or means for manipulation. The '628 patent is clearly directed for use in a different field than the present invention.

WO 98/42398 is directed to a wire dispenser apparatus. This is a hand-held spool of wire, which functions similarly to U.S. Pat. No. 3,995,628, discussed above.

WO 97/14469 is directed to Forceps for the Surgical Introduction of Catheters and the Like. This is a pair of forceps having a channel to receive wire. This disclosure contains neither suggestion of a wire dispenser nor any reference at all to the source of the wire.

These prior art devices are cumbersome and awkward to use. They are especially ill-suited for use in a patient's mouth. The present invention provides an elegant solution to the problem of glove perforation as well as an improvement to the customary surgical procedures using wire.

### SUMMARY OF THE INVENTION

The present invention describes a device which comprises a cartridge which attaches to a pair of forceps which provides a continuous source of wire for surgical procedures, a mount for attaching the cartridge to the forceps, and means for covering the exposed end of the wire, thereby eliminating the passage of wire pieces from the assistant to the surgeon.

It is an object of the present invention to provide an improved system for surgical procedures which require wiring together bone fragments or teeth for orthopedic, maxillofacial or reconstructive surgery.

It is a further object of the present invention to provide an improved method for preventing or minimizing perforation of surgical gloves during reconstructive, maxillofacial or orthopedic surgery.

It is a further object of the present invention to ide an improved device for supplying wire during orthopedic, maxillofacial or reconstructive surgical procedures.

It is a still further object of the present invention to provide a method of reducing assistant participation during orthopedic, maxillofacial or reconstructive surgical procedures.

It is a still further object of the present invention to provide a method of reducing wastage of surgical wire or suture.

It is a still further object of the present invention to provide a device which is capable of delivering more precise lengths of wire or suture for surgical procedures than prior art methods.

It is a still further object of the present invention to provide an easily sterilizable wire dispenser for dispensing surgical wire or suture.

### BRIEF DESCRIPTION OF THE DRAWINGS

FIG. 1A is a side view of the cartridge of the present invention.

FIG. 1B is a cross-sectional view showing the engagement of the sheath to the cartridge along A-A.

FIG. 2A is a top view of forceps showing the mount and wire guide of the present invention.

FIG. 2B is a side view of forceps showing groove.

FIG. 3A is a side view of one half of the cartridge of the present invention without the sheath and showing the channel or travel of the tab of the sheath.

FIG. 3B is an end view showing both halves of the cartridge fitted together.

FIG. 4 shows how the device of the present invention is mounted onto a pair of forceps.

FIG. 5 shows the device of the present invention mounted on a pair of forceps with the sheath retracted exposing the wire.

FIG. 6 shows the device of the present invention mounted on a pair of forceps with the wire exposed and extended through the guide.

FIG. 7 shows a kit containing the cartridge and forceps of the present invention and a wire cutter for one-step sterilization for surgical procedures.

FIG. 5A through FIG. 8C show a pinch-clip alternative mounting means.

FIG. 8B shows an end view of the cartridge of the present invention having a pinch-clip alternative mounting means.

FIG. 8C shows a bottom view of the cartridge of the present invention having a pinch-clip alternative mounting means mounted to a pair of forceps.

### DETAILED DESCRIPTION

Turning now to FIG. 1, the device of the present invention consists of three components. The first component is cartridge 10 containing surgical wire 12 or suture material coiled around a hub 4 in an enclosure 16, which enclosure is typically made of high temperature plastic, such as polysulfone or polyetherimide, for example. Plastics suitable for use in the present invention must be able to withstand high temperatures and chemical sterilization agents. Polyetherimide is commonly available from many plastic supply companies, such as G. E. Plastics, Pittsfield, Mass., for example. The surgical wire is typically stainless steel. The enclosure 16 serves to store the wire and protect the user from the ends of the wire. The enclosure is equipped with a retractable sheath 22 to cover the end of wire 12 where wire 12 exits the enclosure. The two halves are typically designed with holes 24 for circulation of sterilization medium such as steam. The wire is typically stainless steel, for example, annealed type 316LVM stainless steel, gauge 22, 24, or 26.

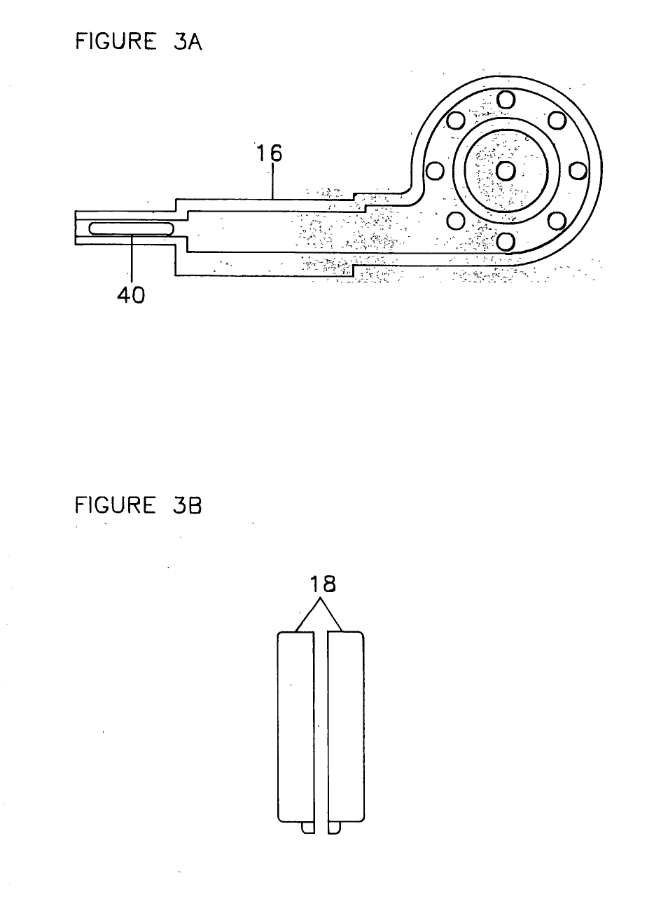

FIGS. 3A and 3B show the enclosure 16, which is typically made in two halves 18 fitted together in a clamshell-type configuration. FIG. 3A is a side view of the cartridge and FIG. 3B is a rear view of the cartridge. The two-halves 18 can be fitted together by any means suitable for this purpose such as an interference fit, a taper-lock fit or a snap fit, or a screw 20, for example. The cartridge is intended for single use. However the cartridge can also be cleaned, sterilized, refilled with wire and re-sterilized for repeated use.

Turning back to FIG. 1A, cartridge 10 comprises channel 40 which receives tab 37 on sheath 22 which prevents sheath 22 from slipping off cartridge 10. Tab 37 travels in channel 40 to retract sheath 22 thus exposing the end of wire 12 for use and extend the sheath to cover the end of wire 12 when not in use. These features ate shown in FIG. 1A.

Sheath 22 is typically a metal tubular element having two, longitudinal slits 41 cut into its outer wall at the rim where the cartridge meets the sheath. During assembly, the material between the two slits is bent toward the center of the sheath to form tab 37. Tab 37 travels in channel 40 to prevent the sheath from sliding completely off of the cartridge 10.

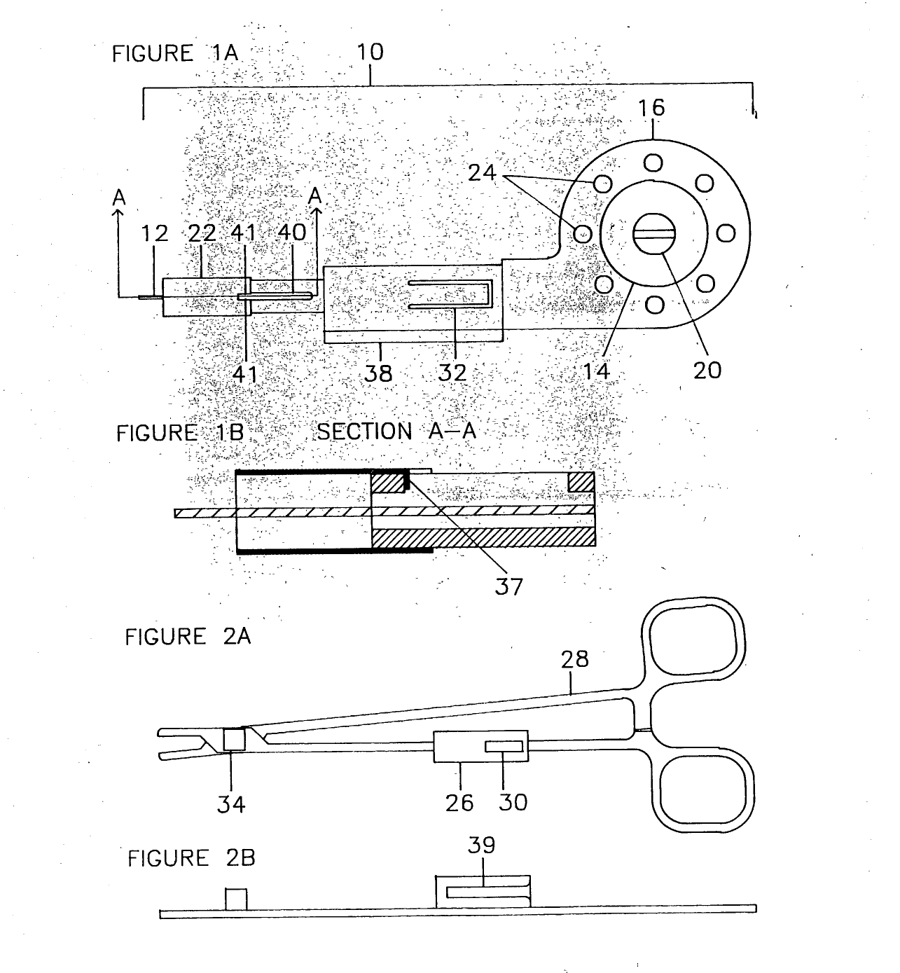

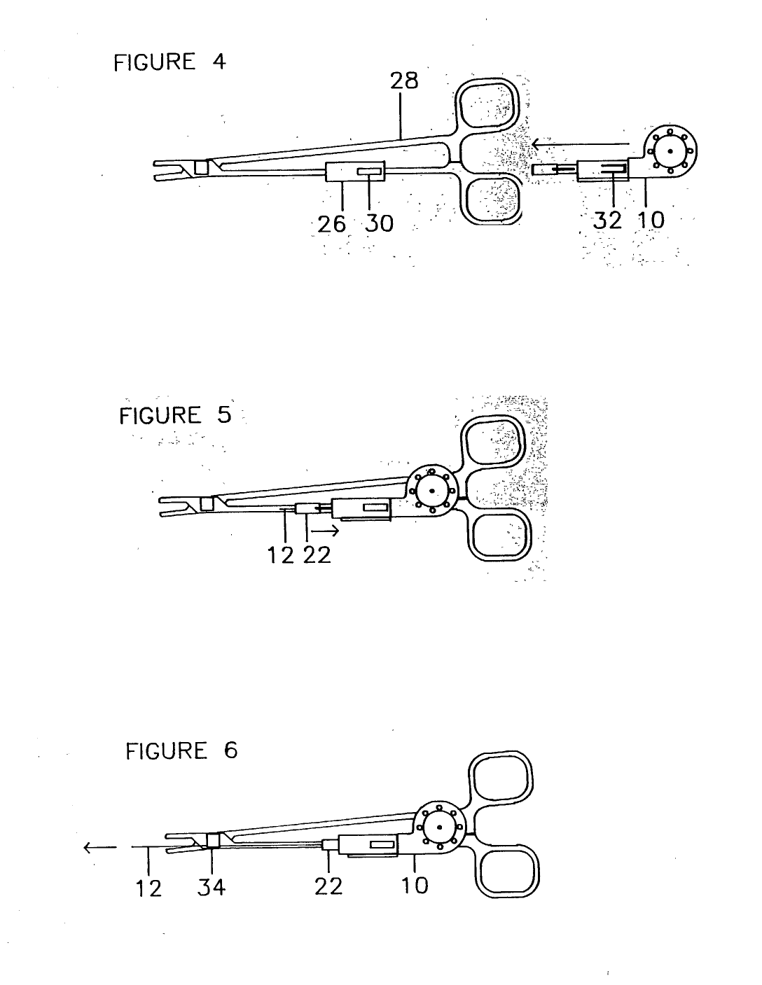

Turning now to FIGS. 2 and 4, the second component of the present invention is a mount 26 which fits onto one side arm of a pair of forceps 28. The mount 26 receives the cartridge 10 and comprises a slot 30 for receiving a tab 32 (shown in FIG. 1) on the cartridge 10 to lock the cartridge 10 in place on the forceps 28. Cartridge 10 may include a 38 to facilitate alignment of the cartridge onto the mount 26. Rib 38 slidably engages into groove 39 on mount 26, shown in FIG. 2B. The mount 26 is positioned on the arm of the forceps so as not to interfere with the handling of the forceps or visualization of the surgical field. The mount 26 may be permanently attached to forceps 28 by welding or other means or mount 26 may be removable from the forceps and attached to the forceps by use of a clip or other attachment mechanism.

An alternative mount is shown in FIGS. 8A, 8B and 8C. The mount of FIG. 8 comprises a pinch-style clip which has two opposable flexible arms 36 which deflect to receive one arm of the forceps 28 and contract to form a pressure fit around the arm of the forceps. FIG. 8B shows an end view of the cartridge of the present invention having a pinch-clip alternative mounting means. FIG. 8C shows a bottom view of the cartridge of the present invention having a pinch-clip alternative mounting means mounted to a pair of forceps. The pinch-clip mount shown in FIG. 8C has the added feature of permitting the use of commercially available, non-modified forceps with the cartridge of the present invention.

Other mounting means obvious to the skilled artisan may also be adapted for used in securing the cartridge to the forceps.

The third component of the present invention is a pair of forceps 28 which can be specially adapted for use with the present invention, as shown in FIG. 2. Forceps so adapted will include a wire guide 34 near the jaws of the forceps 28 for receiving the wire 12. The guide 34 can be a tubular member mounted on the forceps near the jaw of the forceps or a grooved channel cut into the length of the arm of the forceps. Both entry and exit rims of the guide should also be rounded or smoothed so as to prevent damage to the surface of the wire. Although specially adapted forceps can be used in conjunction with the present invention, such specially adapted forceps are not required. Any suitable forceps can be used with the cartridge of the present invention.

In use, the components are sterilized. Sterilization can be accomplished by exposing the components to steam autoclave up to 320° F. or 160° C., ethylene oxide, gamma radiation, or cold liquid sterilants. A separate sterile wire cutter 35 is also provided. The components can be sterilized together in a case provided for this purpose. Such a kit is depicted in FIG. 7.

In use, the mount 26 is affixed to the forceps and the device is assembled by inserting the cartridge 10 into the mount 26 until tab 32 snaps into the slot 30 thereby securing the cartridge 10 onto forceps 28. This is depicted in FIG. 4.

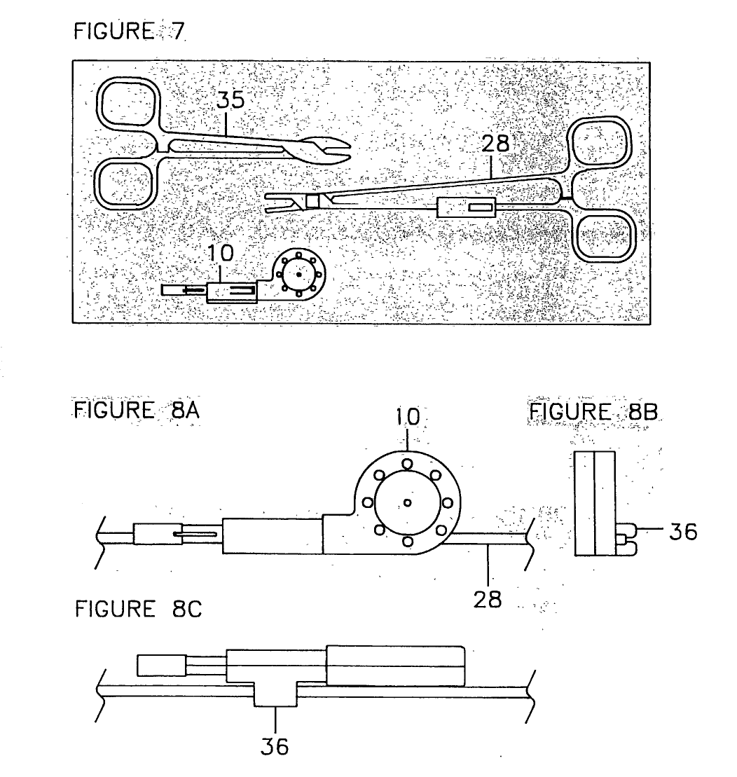

The sheath 22 is then manually retracted thereby exposing the end of the wire 12. The rim the sheath 22 from which the wire exits is rounded or smoothed to prevent damage to the wire. The wire 12 is manually drawn from the cartridge 10 and passed through the guide 34 to expose the desired length of wire 12. See FIG. 6. The wire 12 is then used in the usual manner, such as wrapping around a tooth or bone fragment, and cut with the wire cutter. When the procedure is complete, the exposed end of wire 12 is pushed back into the cartridge 10 and the sheath 22 is manually slid over the end of the wire 12, as shown in FIG. 5.

The cartridge 10 is then removed from the mount 26 by pressing the tab 32 into the slot 30 and pulling the cartridge 10 off of the mount 26. The used cartridge 10 is then disposed of according to appropriate guidelines. If the cartridge is to be re-used, it is cleaned, sterilized, refilled with wire and re-sterilized for use.

Although this invention has been described with respect to specific materials and embodiments, it is not intended to be limited thereto and certain modifications and substitutions will become apparent to the person of ordinary skill in the art, such modifications and substitutions are intended to fall within the spirit and scope of the invention as described is claimed.

1. A wire dispensing device for use with forceps comprising:
A reusable or disposable cartridge comprising two halves which fit together forming a hub to receive a coil of wire having a first end;
a retractable sheath for receiving the first end of the wire positioned at the place of exit of the wire from the cartridge; and mounting means for mounting the cartridge onto a pair of forceps.

2. (canceled)

3. (canceled)

4. The device of claim 1 wherein the mounting means comprises a mount which attaches to the arm of a pair of forceps which mount has a slot for receiving a tab on the cartridge.

5. The device of claim 4 wherein the two halves of the cartridge are fastened together by means of a screw.

6. (canceled)

7. A kit containing the wire dispensing device of claim 1, forceps, and a wire cutter.

8. The device of claim 1 wherein the mounting means comprises a pinch-dip which has two opposable flexible arms which deflect to receive one arm of forceps and contract to form a pressure fit around the arm of the forceps.

9. The device of claim 1 wherein the cartridge and sheath comprise means for preventing the sheath from sliding completely off of the cartridge.

10. The device of claim 9 wherein the means for preventing the sheath from sliding completely off of the cartridge is a tab on the sheath which can be deformed to travel in a channel on the cartridge.

11. The device of claim 4 wherein the mounting means further comprises a rib on the cartridge which fits into a groove on the mount.
